(19) United States
(12) Patent Application Publication
(10) Pub. No.: US 2026/0355365 A1
(43) Pub. Date: Nov. 12, 2026

(54) ANTI-HUMAN ICOS VHH AND USE THEREOF

(71) Applicant: THE FIRST AFFILIATED HOSPITAL OF NINGBO UNIVERSITY, Ningbo (CN)

(72) Inventors: Zunyu XIAO, Ningbo (CN); Weiyu CHEN, Ningbo (CN); Chao LI, Ningbo (CN); Shao DUAN, Ningbo (CN)

(73) Assignee: THE FIRST AFFILIATED HOSPITAL OF NINGBO UNIVERSITY, Ningbo (CN)

(21) Appl. No.: 19/714,627

(22) Filed: Jun. 22, 2026

Related U.S. Application Data
(63) Continuation-in-part of application No. PCT/CN2024/091440, filed on May 7, 2024.

(30) Foreign Application Priority Data
Jan. 25, 2024 (CN) 202410102014.8

(51) Int. Cl. A61K 51/10; A61P 35/00; C07K 16/28

(52) U.S. Cl. CPC A61K 51/1027; A61K 51/1093; A61P 35/00; C07K 16/2818; C07K 2317/569

(57) ABSTRACT

Provided are a human ICOS (Inducible T-cell CO-Stimulator) VHH and a use thereof. The VHHs can bind to human ICOS with high affinity and be used in PET imaging targeting activated T cells.

### CROSS REFERENCE TO RELATED APPLICATIONS

This application is a continuation-in-part (CIP) application based upon International Patent Application No. PCT/CN2024/091440 filed May 7, 2024, which claims the priority of Chinese Patent Application No. 202410102014.8, filed with the China National Intellectual Property Administration on Jan. 25, 2024, which is hereby incorporated by reference in its entirety.

### SEQUENCE LISTING

This application contains references to amino acid sequences and/or nucleic acid sequences which have been submitted concurrently herewith as the sequence listing text file entitled “260051-APXU-FAHNU-Sequence-Listing.xml”, file size 25,153 bytes, created on Jun. 4, 2026. The aforementioned sequence listing is hereby incorporated by reference in its entirety pursuant to 37 CFR 1.835(b)(2).

### FIELD

The present disclosure belongs to the field of immune technology and relates to a VHH (Variable Heavy domain of Heavy chain) targeting human ICOS (Inducible T Cell Costimulator) and use thereof.

### BACKGROUND

Due to the influence of tumor microenvironment heterogeneity, the therapeutic efficacy of immunotherapy varies greatly among different patients, and how to achieve precise prediction/evaluation of therapeutic efficacy in patients is a major issue urgently to be solved. Current nuclear medicine molecular imaging probes used for such studies include:

Metabolic small-molecule probes: in order to achieve precise in vivo prediction and evaluation of lung cancer immunotherapy, researchers have begun to attempt to apply PET imaging to evaluate populations receiving immunotherapy, hoping to establish corresponding diagnostic criteria. ¹⁸F-FDG based on glucose metabolism is currently the most commonly used PET/CT small-molecule probe in clinical practice. Researchers have made preliminary attempts to apply ¹⁸F-FDG imaging in evaluating the therapeutic efficacy of lung cancer immunotherapy. In some patients in whom immunotherapy is effective, ¹⁸F-FDG exhibits high uptake, thereby achieving, to a certain extent, in vivo judgment of the therapeutic efficacy of immunotherapy; however, it has certain limitations, mainly for two reasons: because both tumor cells and activated T cells in the tumor microenvironment are in a state of rapid proliferation, both exhibit high uptake of ¹⁸F-FDG, which makes the two indistinguishable in PET/CT imaging; immunotherapeutic drugs can activate T cells in tumor lymph nodes, and the activated T cells proliferate extensively, causing high uptake of ¹⁸F-FDG, while tumor lymph node metastasis also presents high uptake of ¹⁸F-FDG, such that these two completely different biological processes cannot be distinguished by ¹⁸F-FDG PET imaging. In addition, the small-molecule probe ¹⁸F-AraG based on nucleic acid metabolism has also been applied to imaging studies of activated T cells. Compared with ¹⁸F-FDG, it greatly reduces uptake by tumor cells and increases uptake by immune cells inside tumors, but in addition to activated T cells, ¹⁸F-AraG is still taken up by macrophages, dendritic cells, and B cells, causing non-specific imaging. Thus, due to a lack of specificity, small-molecule metabolic probes cannot precisely reflect the therapeutic efficacy of immunotherapy.

Imaging targeting T cells of different phenotypes: in 2014, the Griessinger team used ⁶⁴Cu to label a T cell receptor (TCR)-targeting antibody and completed in vivo tracing of T cells through PET/CT molecular imaging; the Richard team published articles in PNAS and Cancer Research in 2014 and 2016, respectively, reporting their team's work of using ⁶⁴Cu and ⁸⁹Zr to label CD8-targeting monoclonal antibodies and conducting in vivo imaging studies of CD8⁺ effector T cells; subsequently, Kristensen et al. reported work involving therapeutic efficacy evaluation studies of a Sym021 (human-mouse cross-reactive PD-1 inhibitor) treatment model using CD4⁺ and CD8⁺ dual-molecular probes. However, in solid tumor microenvironment, there are large numbers of exhausted T cells expressing TCR, CD3, and CD8, and molecular imaging targeting T cell phenotypes cannot distinguish exhausted T cells from activated T cells.

Activated T cell-specific targeted imaging: neither metabolic small-molecule probes nor molecular imaging targeting different T cell phenotypes can precisely identify activated T cells in vivo, and the crux lies in the relatively low specificity of the selected targets or metabolic pathways for activated T cells.

### SUMMARY

In view of the above deficiencies in the prior art, the technical problem to be solved by the present disclosure lies in providing an anti-human ICOS VHH (also named human ICOS-targeting nanobody) and use thereof.

An objective of the present disclosure is to address the above problems existing in the prior art by providing a human ICOS-targeting nanobody and use thereof. Inducible T-cell CO-Stimulator (ICOS), also known as CD278, is an important immune co-stimulatory receptor, belongs to the CD28 family, and plays a key regulatory role in adaptive immune responses.

In one aspect, the present disclosure provides a polypeptide that specifically binds human ICOS, comprising at least one VHH (Variable Heavy domain of Heavy chain), wherein the VHH comprising CDR1, CDR2, and CDR3, and a combination of the CDR1, CDR2, and CDR3 is selected from the group consisting of:
(a) a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
(d) a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22; and
(e) a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25.

In some embodiments, the polypeptide comprises an amino acid sequence selected from the group consisting of SEQ ID NO: 6, SEQ ID NO: 7, SEQ ID NO: 8, SEQ ID NO: 9, and SEQ ID NO: 10.

In some embodiments, the polypeptide comprises at least one VHH, wherein the VHH comprising CDR1, CDR2, and CDR3, the CDR1 comprising the amino acid sequence set forth in SEQ ID NO: 11, the CDR2 comprising the amino acid sequence set forth in SEQ ID NO: 12, and the CDR3 comprising the amino acid sequence set forth in SEQ ID NO: 13.

In some embodiments, the polypeptide comprises the amino acid sequence set forth in SEQ ID NO: 6.

In another aspect, the present disclosure provides a polynucleotide encoding the polypeptide as described above.

In some embodiments, the polynucleotide comprises a sequence selected from the group consisting of SEQ ID NO: 1, SEQ ID NO: 2, SEQ ID NO: 3, SEQ ID NO: 4, and SEQ ID NO: 5.

In another aspect, the present disclosure provides an expression vector comprising the polynucleotide as described above.

In another aspect, the present disclosure provides a conjugate comprising the polypeptide as described above, wherein the polypeptide is conjugated to a radionuclide.

In some embodiments, the radionuclide is ⁶⁸Ga.

In another aspect, the present disclosure provides a pharmaceutical composition comprising the conjugate as described above and a pharmaceutically acceptable carrier.

In another aspect, the present disclosure provides a method for detecting or imaging activated T cells in a sample or a subject in need thereof, comprising:
administering the conjugate as described above; and
detecting a signal from the radionuclide.

In another aspect, the present disclosure provides a method for treating a tumor, comprising administering to a subject in need thereof a therapeutically effective amount of the polypeptide as described above.

Compared with the prior art, the present disclosure has the following beneficial effects: by independently developing a human ICOS-targeting nanobody, the present disclosure solves the problems in conventional technologies in which an ICOS molecular probe constructed based on a monoclonal antibody has a large molecular weight (approximately 150000), an excessively long in vivo circulation time, excessively high liver uptake, and poor tissue permeability, and also solves the problem that the Fc segment of the monoclonal antibody binds to Fc receptors on macrophages and dendritic cells, causing non-specific uptake and greatly reducing the accuracy of imaging results.

### BRIEF DESCRIPTION OF DRAWINGS

FIG. 1 is a schematic diagram of construction of a gene library in Example 1, in which the left panel shows 150 bp and 200 bp gene fragments, and the right side shows a 330 bp gene fragment;

FIG. 2 is a result diagram of flow cytometry validation of c-myc expression in Example 1;

FIG. 3 is a schematic diagram of the exponential decay pulse waveform during the electroporation process in Example 1;

FIG. 4 shows validation results of colony PCR in Example 1;

FIG. 5 shows results of restriction enzyme digestion verification in Example 1;

FIG. 6 shows sequencing results of Example 1;

FIG. 7 shows validation results of binding affinity of ICOS nanobodies;

FIG. 8 is a schematic diagram of synthesis and characterization of [⁶⁸Ga]Ga-NOTA-ICOS-53 in Example 6, in which FIG. 8A shows median fluorescence intensity (MFI) of different cells; FIG. 8B shows the synthesis process of [⁶⁸Ga]Ga-NOTA-ICOS-53; and FIG. 8C shows radiochemical purity analysis results of [⁶⁸Ga]Ga-NOTA-ICOS-53 and proportions of radioactive cells among different cells;

FIG. 9 shows PET/CT imaging results of [⁶⁸Ga]Ga-NOTA-ICOS-53 in different tumor models, in which FIG. 9A shows PET images; FIG. 9B shows tracer uptake of different tumors at different time points; and FIG. 9C shows the time-course pharmacokinetic curve of [⁶⁸Ga]Ga-NOTA-ICOS-53;

FIG. 10 shows ex vivo biodistribution and immunofluorescence staining results of [⁶⁸Ga]Ga-NOTA-ICOS-53, in which FIG. 10A shows accumulation of [⁶⁸Ga]Ga-NOTA-ICOS-53 in different tissues; FIG. 10B shows correlation between biodistribution quantitative values and PET ROI measurement values; and FIG. 10C shows immunofluorescence staining results of different tumors.

### DETAILED DESCRIPTION

To further describe the technical solutions of the present disclosure, specific examples of the present disclosure are provided below, however, the present disclosure is not limited to these examples.

### Example 1

First, through steps S1 and S2, ICOS antigen was expressed and further purified, thereby providing an antigen basis for subsequent antibody screening.

### S1. ICOS Protein Antigen Expression

Experimental purpose: the purpose of this step was to prepare a target antigen for subsequent nanobody screening. Specifically, ICOS protein with a His tag was expressed using a prokaryotic expression system to provide a raw material for protein purification in the next step.
Prokaryotic expression vector: pET; expression host: BL21(DE3)
ORF: ICOS-poly his

The target protein was expressed using E. coli Strain BL21(DE3) competent cells: after identified clones were cultured, recombinant plasmids were extracted; then the recombinant plasmids were transformed into E. coli Strain BL21(DE3) competent cells, followed by plasmid extraction and transformation.

### Protein Expression and Extraction:

(1) E. coli Strain BL21(DE3) comprising the recombinant plasmid was inoculated into 5 ml of LB medium containing an antibiotic and cultured overnight at 37 °C and 200 rpm. The overnight culture medium was inoculated into 500 ml of LB medium containing an antibiotic and cultured to OD600=0.4 to 0.6.
(2) IPTG was added to induce target protein expression. For example, the concentration of IPTG may be 0.05 to 1.0 mM, and the induction time may be 1 to 5 h.
(3) E. coli cells were collected by centrifugation (16000 g for 3 min), and the cell pellet was resuspended in lysis buffer (100 μl of 50 mM Tris-HCl buffer, pH 8.0, containing 200 mM NaCl, 2% Triton X-100 and 3 mM protease inhibitor PMSF), and then sonicated on ice 20 times (sonicated 3 s with 7 s intervals between each pulse).
(4) The sonicated cells were centrifuged (16000 g for 15 min at 4 °C), and a portion of the supernatant was mixed with 6× loading buffer and heated at 95 °C for 5 min. SDS-PAGE was used for gel electrophoresis, and staining was performed with Coomassie Brilliant Blue R250, whereby successful expression of the target protein was detected.

### S2. Purification of ICOS Protein Using His-Tag Purification

Experimental purpose: in this step, high-purity ICOS protein was separated and purified from the crude protein extract obtained in step S1 by using the His tag in column chromatography, for subsequent biotinylation and screening.

The column chromatography method and reagents used were as follows:
Binding buffer: 20 mM sodium phosphate buffer, 0.5 M sodium chloride, 20 to 40 mM imidazole, pH 7.4;
Elution buffer: 20 mM sodium phosphate buffer, 0.5 M sodium chloride, 500 mM imidazole, pH 7.4;

The sample was filtered before loading, and the buffers were pre-filtered and sonicated to remove bubbles.
(1) A low flow rate was set for the system, the plug at the top of the prepacked column was removed, and the column was connected to the connector of the ÄKTA purifier, with attention to droplet-to-droplet connection to prevent introduction of bubbles.
(2) The plug at the bottom of the column was removed, and the column was connected to the purifier.
(3) Ethanol was washed away with 3 to 5 column volumes of distilled water.
(4) The column was equilibrated with 5 column volumes of binding buffer. For a 1 ml column, the equilibration flow rate was 1 ml/min; and for a 5 ml column, the equilibration flow rate was 5 ml/min.
(5) A pretreated sample was loaded using a sample loop or Superloop (the sample was centrifuged and filtered before column loading). For a 1 ml column, the loading flow rate was 0.2 to 1 ml/min; and for a 5 ml column, the loading flow rate was 0.5 to 5 ml/min.
(6) The column was washed with binding buffer for at least 10 to 15 column volumes until the absorbance peak reached a stable baseline or no fragment flowed out in the effluent. For a 1 ml column, the washing flow rate was 1 to 2 ml/min; and for a 5 ml column, the washing flow rate was 5 to 10 ml/min.
(7) Elution was performed using elution buffer. The elution may employ one-step elution or linear gradient elution (adjusting the imidazole concentration gradient). For one-step elution, 5 column volumes of elution buffer were used; and for linear elution, 10 to 20 column volumes of elution buffer were used. For a 1 ml column, the elution flow rate was 1 to 2 ml/min; and for a 5 ml column, the elution flow rate was 5 to 10 ml/min.
(8) After elution, the column was washed with 3 to 5 column volumes of binding buffer, and then 20% ethanol was added.

### S3. Construction of Yeast Surface Display (YSD) Library NL540

Experimental purpose: in this step, an alpaca was immunized, and then a resulting VHH gene library was transformed into yeast to establish a yeast display library for subsequent screening of nanobodies that specifically bind ICOS.

Before alpaca immunization, 10 mL of blood was collected as a negative serum control. 0.5 mg of recombinant ICOS extracellular domain protein was subcutaneously injected into a healthy male alpaca once every two weeks for a total of 5 times. After the fifth immunization, peripheral blood was collected and blood lymphocytes were isolated. B-cell mRNA was reverse-transcribed into DNA, and a yeast display library for ICOS nanobody screening was subsequently established.

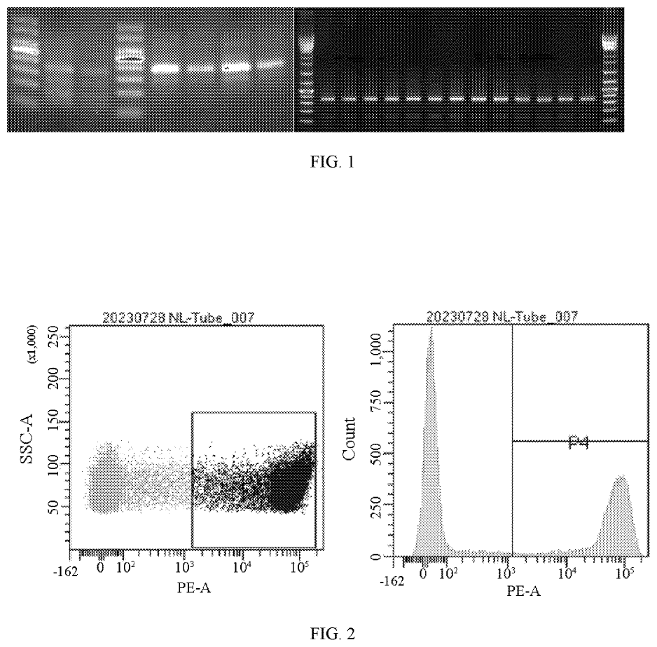

Identification results of the VHH gene library are shown in FIG. 1. The VHH gene library was transformed into yeast cells and subjected to homologous recombination with a vector having Aga2 protein at the N terminus for yeast surface display and a c-myc epitope at the C terminus (for detecting full-length VHH), thereby obtaining 8×10⁷ yeast transformants. Cells expressing c-myc were detected by flow cytometry to validate VHH expression (results are shown in FIG. 2).

### (1) Steps of Electroporation Transformation:

(1) EBY100 yeast cells were cultured overnight in YPD medium. 1×10⁸ cells (OD=0.1) were inoculated into 100 mL of YPD. The cells were cultured at 30 °C and 250 rpm until the cell density was 1.3 to 1.5×10⁷ cells/mL.
(2) The above culture at 30 °C and 250 rpm generally required 6 to 8 hours. If necessary, inoculation may be performed at a lower density and culture may be performed for a longer time, for example overnight, and the cultured cells were subsequently used to prepare competent cells.

### (3) Preparation of Competent Cells:

Electroporation cuvettes and E buffer were cooled, 25 ml LiAC was added to every 50 ml of 1.3 OD EBY100 cells, centrifugation was performed at 3000 rpm for 3 minutes, and the supernatant was removed;

The pellet was resuspended in 25 mL of a solution comprising 100 mM lithium acetate, 10 mM Tris (pH 7.5), and 1 mM EDTA; the mixture was incubated at 30 °C for 15 to 60 minutes and then gently shaken (110 rpm); 0.096 g DTT was added to 0.5 mL 1 M Tris, pH 8.0, filter-sterilized, and then added to the EBY100 cell culture system; incubation was performed at 30 °C and 250 rpm for 15 minutes; centrifugation was performed at 2500 g and 4 °C for 3 minutes; the supernatant was discarded; the cells were washed and resuspended with 25 mL of E buffer (10 mM Tris, pH 7.5, 270 mM sucrose, 1 mM MgCl₂); centrifugation was performed at 2500 g and 4 °C for 3 minutes. The supernatant was discarded; the cells were resuspended in 1 mL of E buffer. The cells were transferred into a 1.5 mL tube; centrifugation was performed at 5000 g and 4 °C for 1 minute; the supernatant was discarded; the cells were resuspended in 1 mL of E buffer; centrifugation was performed at 5000 g and 4 °C for 1 minute. The supernatant was discarded; and the cells were resuspended to a total volume of 300 μL.

### (4) Electroporation:

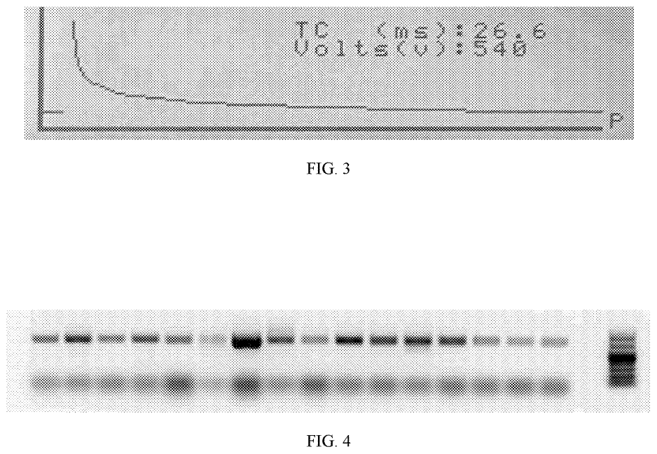

Samples were prepared in microcentrifuge tubes: 150 μL cells+2 μL vector (4 μg)+insert DNA (10 μg); the mixture was transferred into two electroporation cuvettes and incubated on ice for approximately 5 minutes; electroporation was performed using a pulse of 25 μF and 0.54 kV (for a 2 mm cuvette); the time constant was 15 to 45 ms. The exponential decay pulse waveform during the electroporation process is shown in FIG. 3. 1 to 2 mL of room-temperature YPD was added into the electroporation cuvettes. The mixture was transferred into a 14 mL Falcon tube; incubation was performed at 30 °C and 250 rpm for 1 to 2 hours; centrifugation was performed at 1300 g for 1 minute; the cell pellet was resuspended in 1 mL of SD-CAA and then transferred into a flask containing 50 to 1000 mL of SD-CAA. As shown by FIG. 3, the TC time of the exponential decay pulse waveform conformed to the optimal perforation time for yeast electrotransformation, and under this transformation constant, the library construction transformation efficiency was highest.

### S4. Yeast Growth and Induction:

In this step, the yeast library NL540 constructed in step S3 was subjected to expansion and induced expression, and the constructed yeast library was identified.

### Expansion Culture:

Day 1: at 5:00 p.m., the cells were thawed and inoculated into a 2 L flask with a stir bar containing 1 L of S(D)CAA P/S, and maintained at 4 °C for a period of time. The density of the cells should be less than 2.5×10⁶ cells/mL. The cells were cultured overnight at 30 °C with stirring at 250 rpm. (Culture was started from an OD of 0.1 to 0.2);
Day 2: at 8:30 a.m., the cells were counted (using a hemocytometer), and the cells were diluted 10-fold to adjust the density to 2×10⁶ cells per milliliter, and inoculated in prewarmed 30 °C S(D)CAA+P/S at at least 10 times the library diversity (if an original library was used, 20 times was used for a library with diversity less than 2×10⁷). The cells were inoculated into a 2 L flask with a stir bar containing 1 L of S(D)CAA P/S.

The cells were cultured at 30 °C and 250 rpm for 7 hours (ensuring that, after culture, the cells were in logarithmic growth phase and the size of the original cells was approximately twice that of dividing cells) (after 7 hours, the OD should not exceed 3);

Induced expression culture: after 7 hours of growth, cell density was measured (hemocytometer). 6×10⁹ cells were harvested in a 50 ml conical centrifuge tube and centrifuged at 2500 g for 5 minutes, the supernatant was discarded, and the cells were resuspended and transferred into 300 mL of prewarmed S (G) CAA+P/S (galactose yeast nutrient medium containing final concentrations of 2% galactose and 0.1% glucose), the density was adjusted to 2×10⁷/mL, and the cells were transferred into a 1 L flask with a stir bar. The cells were cultured at 30 (or 20)°C and 250 rpm for at least 16 hours.

The S(D)CAA culture was stored in a cold room until completion of first-round sorting, and the yeast library was identified by colony PCR, restriction digestion, and sequencing, respectively.

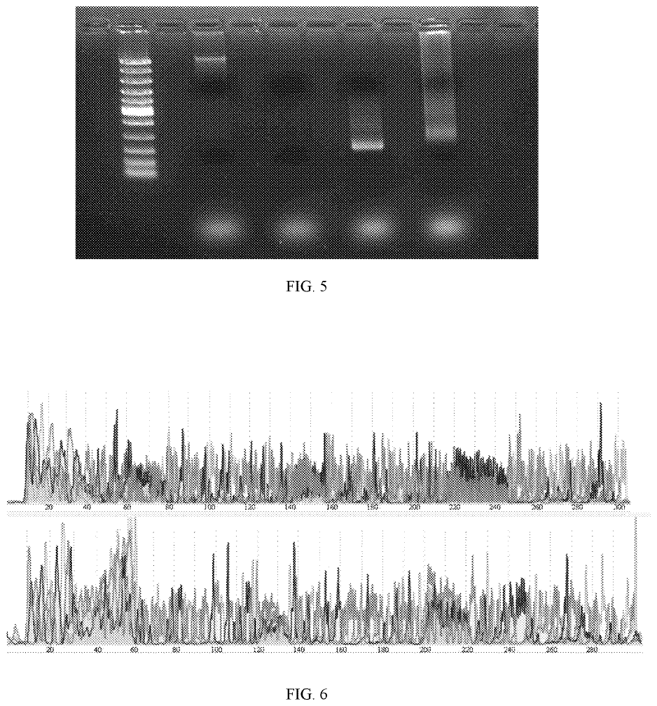

FIG. 4 shows colony PCR validation results, from which it can be seen that the colony positive clone rate reached 100%.

FIG. 5 shows restriction enzyme digestion verification results, from which it can be seen that restriction enzyme digestion verification of extracted yeast plasmids indicated successful first-round library construction of NL540.

FIG. 6 is a diagram of sequencing results, from which it can be seen that sequencing from the 5′ and 3′ ends of the sequence showed that three variable regions were present in the middle, the distribution frequencies of the four types of bases were identical, and the constant framework regions at the four ends were consistent with the VHH domain sequence, thereby verifying the accuracy of library construction.

The results demonstrate that the yeast library NL540 was successfully constructed.

### S5. Naïve Yeast Library and Culture Conditions

This step established standard operating parameters required for maintaining and culturing a Naïve yeast library. By specifying medium types, culture temperature, pH value, and antibiotic selection strategy, a suitable environmental basis was provided for normal growth of yeast cells, protein expression, and library stability.

Medium selection: according to experimental requirements, media including YPD, SDCAA, and SGCAA and corresponding plates were selected.

Temperature: generally, the culture temperature for yeast surface display experiments may be maintained between 28 °C and 30 °C, which is ideal for yeast growth and protein expression.

pH value: the pH value of the medium may be controlled between pH 5.5 and 6.5, which is conducive to yeast cell growth and protein expression.

Antibiotic selection: by utilizing the ampicillin antibiotic resistance possessed by the yeast strain, growth of contaminating bacteria was inhibited using an antibiotic selection method.

### S6. Screening of ICOS Nanobodies

Experimental purpose: starting from a naive yeast library, high-affinity ICOS-specific nanobodies were screened in this step, purified nanobodies were then obtained by prokaryotic expression, their specific binding to activated T cells was verified, and finally a radionuclide-labeled probe was prepared.

The initial yeast library was cultured and subjected to induced expression and magnetic bead screening to reduce nonspecific binding and improve efficiency of binding to the biotinylated antigen. The resulting cells were further enriched by FACS to select high-affinity cells and for analytical monitoring. Plasmids were extracted from the enriched cells, their sequences were analyzed, and dominant sequences were retransformed into yeast for binding characterization at the monoclonal level. After clones of interest were identified, random mutations were introduced by error-prone PCR, and a secondary mutant library was constructed by yeast homologous recombination. On one hand, yeast carrying high-affinity antibodies was enriched by FACS screening; on the other hand, multiple rounds of iterative FACS screening were performed by kinetic competition for enrichment until the desired affinity was achieved.
(1) Magnetic bead screening: Biotin Binder Dynabead (Invitrogen) was used for initial library screening. The ICOS antigen was subjected to biotinylation treatment before sorting. The initial library was induced for more than 16 h at 30 °C in SGCAA medium, and the biotinylated ICOS antigen was linked to dynabeads.

Negative magnetic bead screening: after yeast cells were incubated with negative magnetic beads at 4 °C for 2 hours, cells bound to the magnetic beads were removed using a magnetic rack, and unbound cells were transferred into a washed tube containing antigen-coated magnetic beads.

Positive magnetic bead screening: yeast cells were cultured with positive magnetic beads at 4 °C for at least 2 hours. Yeast cells were expanded and subjected to induced expression, and the induced yeast cells should be enriched in ICOS/antigen binders. To ensure specificity, washing stringency was increased, and negative selection purification was further performed.

After two rounds of magnetic bead enrichment, double-positive yeast was isolated by FACS, thereby screening out yeast cells displaying full-length VHH clones on the surface.
(2) VHH mutagenesis and electroporation: to reintroduce diversity into the screened VHH binders, plasmids were extracted from yeast and mutagenesis was performed.

Yeast plasmids were extracted, error-prone PCR was performed using 8-oxo-2-deoxyguanosine-5-triphosphate and 2-deoxy-p-nucleoside-5 triphosphate (8-oxo-dGTP and dPTP) as raw materials (reaction conditions are shown in Table 1), and the mutagenized VHH gene sequences were amplified by PCR (reaction conditions are shown in Table 2), so as to generate a sufficient number of DNA inserts for yeast transformation.

TABLE 1
Error-pronePCR

| | Temperature | | |
|---|---|---|---|
| Step | (°C) | Time | Cycle |
| Initial denaturation | 94 | 3 min | 1 |
| Denaturation | 94 | 45 s | |
| Annealing | 60 | 30 s | 2-16 |
| Extension | 72 | 90 s | |
| Final extension | 72 | 10 min | 17 |

TABLE 2
Amplification PCR

| Step | Temperature (°C) | Time | Cycle |
|---|---|---|---|
| Initial denaturation | 94 | 3 min | 1 |
| Denaturation | 94 | 45 s | |
| Annealing | 60 | 30 s | 2-31 |
| Extension | 72 | 90 s | |
| Final extension | 72 | 10 min | 32 |

Using pCT-CON2 as a vector, the amplified gene fragments were mixed with the linearized vector and introduced into wild-type yeast cells by electroporation. Homologous recombination naturally occurred in yeast, causing the linearized vector and inserts to recircularize.

### (3) FACS Screening

After several rounds of magnetic bead enrichment, the yeast-displayed VHH library was quantitatively screened by FACS. Double labeling was performed by c-myc tag detection and binding to soluble antigen. Using this double labeling, collection screening was performed.

In addition, dissociation competition may be used for further screening: cells were first labeled with a stoichiometric excess of biotinylated antigen, washed, and then unlabeled non-biotinylated antigen was added for a competition reaction.

FACS assay was performed on yeast cell populations respectively expressing 97 candidate sequences, and the binding ability of the antibodies to ICOS was evaluated by the following two indicators:
(a) Normalized fluorescence intensity: to exclude differences in yeast surface expression abundance among different clones, a ratio of fluorescence intensity of an antigen-binding signal to an antibody expression signal in a single cell was calculated. The average value of this ratio in a cell population represented normalized fluorescence intensity and reflected the apparent affinity of the antibody;
(b) Percentage of antigen-binding positive cells: among a cell population successfully displaying an antibody (c-myc⁺), the percentage of cells capable of specifically binding the ICOS antigen (c-myc⁺&SA-650⁺), which was used to measure binding stability and abundance of candidate clones at a specific antigen concentration.

The normalized fluorescence intensity of different yeast cell populations was as low as 21.52 and as high as 928.26; the percentage of antigen-binding positive cells of different yeast cell populations was as low as 10.02% and as high as 99.32%. The five VHHs with the highest normalized fluorescence intensity, i.e., the highest affinity, numbered as 225, 321, 131, 53, and 472, were selected for subsequent experiments.

These five sequences were considered to express high-affinity antibodies, and normalized fluorescence intensity and percentage of antigen-binding positive cells of the cells to negative control cells are shown in Table 3:

TABLE 3
Normalized fluorescence intensity and percentage of antigen-binding
positive cells of high-affinity cells and negative control cells

| | Normalized | percentage of |
|---|---|---|
| | fluorescence | antigen-binding |
| VHH No. | intensity | positive cells |
| 225 | 928.26 | 99.32% |
| 321 | 497.49 | 98.35% |
| 131 | 259.45 | 94.03% |
| 53 | 207.03 | 88.48% |
| 472 | 180.22 | 88.99% |

### (4) Monoclonal Identification Analysis

The above high-affinity yeast cells were collected, yeast plasmids were extracted, the plasmids were transferred into DH5a, monoclonal colonies were picked and amplified, bacterial plasmids were extracted, and sequencing was submitted for detection.

### (5) ICOS Nanobody Expression

First, according to the finally collected sequencing sequences, PET-VHH expression vector sequences were constructed, and VHH proteins were expressed using the prokaryotic expression system in S1. After cell lysis, the supernatant was subjected to sonication, and VHH proteins were purified using suitable tag purification according to the purification tag fused to the VHH, for binding verification in (6).

### (6) Flow Cytometric Validation of Selective Binding of ICOS Nanobodies to Activated T Cells

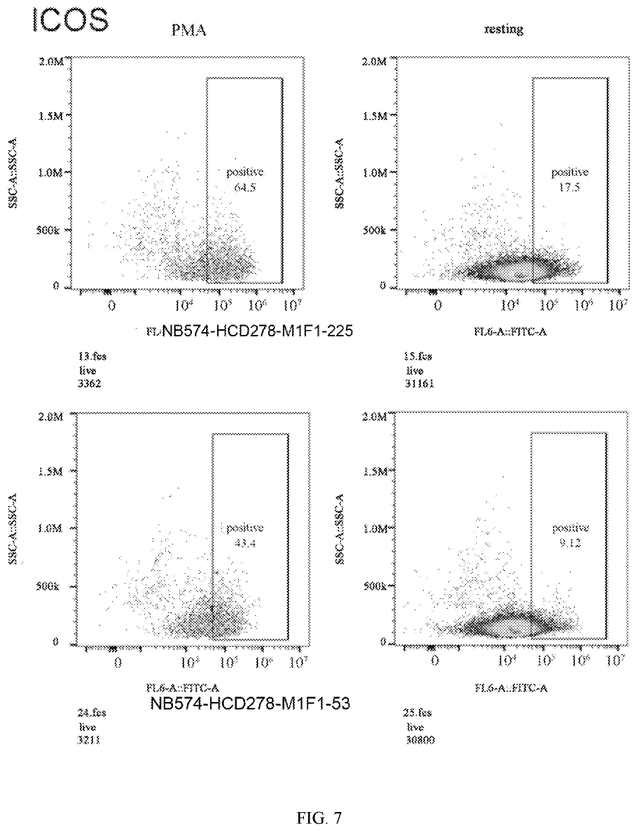

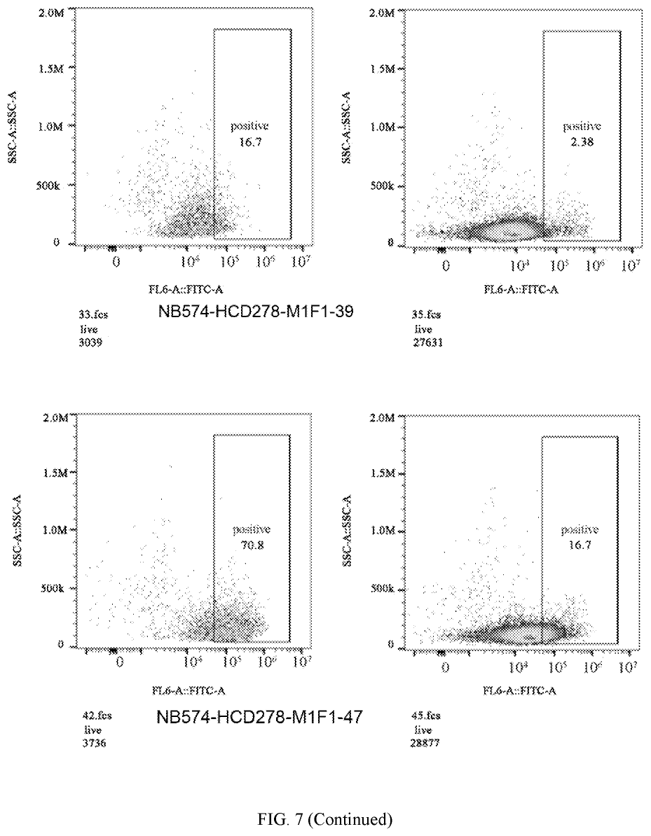

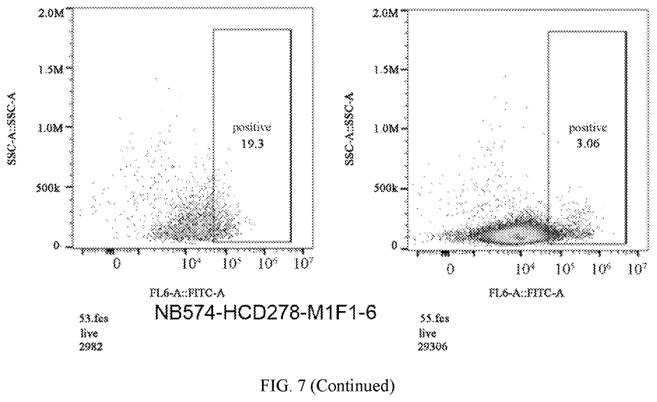

The ICOS nanobodies expressed in (5) were used to verify the specific binding ability of various antibodies to activated T cells.
(a) Cell grouping: Positive cell and Negative cell.
Positive cell: 50 million PBMC cells were recovered and cultured for 24 h, and then PMA/INON was added for stimulation for 48 to 72 h; negative cells: resting PBMC cells;
(b) 5×10⁴ cells were transferred to each well of a U-bottom 96-well plate for flow staining: cells were washed with 200 μL of PBS, then centrifuged at 2000 rpm, the supernatant was discarded, and 1 μL of ICOS nanobody (each sequence was pre-diluted to 1 μg/uL) was added to each well and incubated for 20 min protected from light;
(c) The cells were washed with 200 μL of PBS, centrifuged at 2000 rpm, and the supernatant was discarded; this was repeated once;
(d) 1 μg of secondary antibody was added to each well, and incubation was performed for 20 min protected from light;
(e) The cells were washed with 200 μL of PBS, centrifuged at 2000 rpm, and the supernatant was discarded; this was repeated once;
(f) The cells were resuspended using FACS buffer and detected on the instrument (results are shown in FIG. 7).

It can be seen from FIG. 7 that, for each antibody, in the resting PBMC cell population on the right side, most cells fell within a low fluorescence intensity region, indicating that the nanobody had less binding to T cells in a non-activated state; whereas in the activated PBMC cells on the left side, cells located in the high fluorescence intensity region were significantly increased.

It can be seen that these ICOS nanobodies had significantly higher binding affinity to activated T cells than to resting T cells, could effectively label activated T cells, and could be used for in vivo precise evaluation of lung cancer immunotherapy effects, or used as molecular probes for localization and imaging of immunologically active regions such as autoimmune diseases and infections, and may also be used to deliver immunosuppressants or cytotoxic drugs to T cell populations activated under pathological conditions, thereby improving therapeutic efficacy and reducing side effects.

The gene sequence numbered 225 is shown in SEQ ID NO: 1:
CAGGTGCAGCTCGTGGAGTCTGGGGGAGGCTTGGTGCAGCCTGGGGGTTCT CTGAGACTCTCCTGTGCAGCCTCTGGATTCACTTTTGAGCGTTGGACCATGGGGTGGT TCCGCCAGACCCCAGGGAAGGAGCGCGAGGGGATCTCATGTATTAGTAGTAGTGATG GTAGCACATACTATGGAGACTCCAAATACTATGGAGACTCCGTTCGGGGCCGATTCACT ATTTCCAGAGACAACGCCAAGAACACAGTATATCTGCAAATGAACAACTTGAAACCT GAGGACACGGCCGTTTATTACTGTGCAGCAGCCCAAGGGTATAGCGACTATGGATGCT ACGGCATGGAGTACTGGGGCAAAGGGACCCTGGTCACCGTCTCCTCAT.

The corresponding VHH antibody sequence of the human ICOS-targeting nanobody is shown in SEQ ID NO: 6, and this VHH antibody was denoted as ICOS-53:
QVQLVESGGGLVQPGGSLRLSCAASGFTFERWTMGWFRQTPGKEREGISCISSS DGSTYYGDSKYYGDSVRGRFTISRDNAKNTVYLQMNNLKPEDTAVYYCAAAQGYSDY GCYGMEYWGKGTLVTVSS; and
CDR1 sequence is shown in SEQ ID NO: 11: GFTFERWT;
CDR2 sequence is shown in SEQ ID NO: 12: ISSSDGSTYYGDSK;
CDR3 sequence is shown in SEQ ID NO: 13: AAAQGYSDYGCYGMEY.

A surface plasmon resonance (SPR) assay was performed between ICOS-53 and ICOS extracellular protein using a Biacore T200 system (GE Healthcare). The results showed that ICOS-53 had high binding affinity for the human ICOS extracellular domain, with an equilibrium dissociation constant (KD) of 85.3 pM.

### Example 2

The sequence of the gene numbered 321 in Example 1 is shown in SEQ ID NO: 2:
caggtgcagctcgtggagtccggcggaggcttggtgcagcctggggggtctctgagactctcctgtgcagcctctggaaaca tcttcagtatcaatgccatgggctggtaccgccaggctccagggaagcagcgcgagttggtcgcagcgtttactagtgatggtagcacagac tatggagactccgtgaagggccgattcaccatctctagggacaacgccgagaacacggtggttttgcaaatgaacagcctgaaacctgagg acacggccgtctattactgcaatgcccaagttttctcaaggactatggagggctatgagggcatggagtactggggcaaagggaccctggtc accgtctcctcag.

The corresponding VHH sequence of the human ICOS-targeting nanobody is shown in SEQ ID NO: 7:
QVQLVESGGGLVQPGGSLRLSCAASGNIFSINAMGWYRQAPGKQRELVAAFTSDGSTDY GDSVKGRFTISRDNAENTVVLQMNSLKPEDTAVYYCNAQVFSRTMEGYEGMEYWGKG TLVTVSS; and
CDR1 sequence is shown in SEQ ID NO: 14: GNIFSINA;
CDR2 sequence is shown in SEQ ID NO: 15: FTSDGST;
CDR3 sequence is shown in SEQ ID NO: 16: NAQVFSRTMEGYEGMEY.

### Example 3

The sequence of the gene numbered 131 in Example 1 is shown in SEQ ID NO: 3: caggtgcagctcgtggagtccggcggaggcttggtgcagcctggggggtctctgagactctcctgtgcagcctctggaaacatcttcagtat caatgccatgggctggtaccgccaggctccagggaagcagcgcgagttggtcgcagcgtttactagtgatggtagcacagactatggaga ctccgtgaagggccgattcaccatctctagggacaacgccgagaacacggtggttttgcaaatgaacagcctgaaacctgaggacacggc cgtctattactgcaatgcccaagttttctcaaggactatggagggctatgagggcatggagtactggggcaaagggaccctggtcaccgtct cctcag.

The VHH sequence of the corresponding human ICOS-targeting nanobody is as set forth in SEQ ID NO: 8:
QLQLVESGGGLVQAGGSLRVSCAASGFTFDDYTIGWFRQSPGKEREGVSCISSSVGITYYA DSVKGRFTISSDNAKNTVYLQMNSLKPEDTAVYYCAAGPTDSTLCGSLPDSGSWGQGTQ VTVSS; and
CDR1 sequence is as set forth in SEQ ID NO: 17: GFTFDDYT;
CDR2 sequence is as set forth in SEQ ID NO: 18: ISSSVGIT;
CDR3 sequence is as set forth in SEQ ID NO: 19: AAGPTDSTLCGSLPDSGS.

### Example 4

The sequence of the gene numbered 53 in Example 1 is as set forth in SEQ ID NO: 4:
gaggtgcagctggtggagtctgggggaggcttggtgcagcctgggggttctctgagactctcctgtgcagcctctggagcca ctttggataattatgccacaggctggttccgccaggccccagggaaggagcgcgaggaggtctcatgtataagtggtagtggtgatggtaac acatactatgcagactccgtgaagggccgattcaccgtctccagagacagcgccaagaacacggtgtatctggaaatgaacagcctgaaac cggaggacacggccgtttattactgtgcagccgagggaccatatacatactatagtggtactggctgcccgggcacaacagggtatctcga agtttggggccagggcaccctggtcactgtctcctcag.

The VHH sequence of the corresponding human ICOS-targeting nanobody is as set forth in SEQ ID NO: 9:
EVOLVESGGGLVQPGGSLRLSCAASGATLDNYATGWFRQAPGKEREEVSCISGSGDGNT YYADSVKGRFTVSRDSAKNTVYLEMNSLKPEDTAVYYCAAEGPYTYYSGTGCPGTTGY LEVWGQGTLVTVSS; and
CDR1 sequence is as set forth in SEQ ID NO: 20: GATLDNYA;
CDR2 sequence is as set forth in SEQ ID NO: 21: ISGSGDGNT;
CDR3 sequence is as set forth in SEQ ID NO: 22 AAEGPYTYYSGTGCPGTTGYLEV.

### Example 5

The sequence of the gene numbered 472 in Example 1 is as set forth in SEQ ID NO: 5: gaggtgcagctggtggagtctgggggagacttggcgcaggctggggggtctttgagactctcctgtgcagcctctggattcactttcgatgat tatgtcataggctggttccgccaggccccagggaaggagcgtgagggggtctcatgtatgagtagtagtgatggtataacatactacgcaga ctccgtgaagggccgattcaccatctccagtgacaacgccaagaatacggtgtatctgcaaatgaacagcctgaaacctgaggacacggcc gtttattactgtgcagcgggacccagtgacccaacgctgtgcggtagcttacctgactttggttcctggggccaggggacccgggtcaccgt ctcttcag.

The VHH sequence of the corresponding human ICOS-targeting nanobody is as set forth in SEQ ID NO: 10:
EVQLVESGGDLAQAGGSLRLSCAASGFTFDDYVIGWFRQAPGKEREGVSCMSSSDGITY YADSVKGRFTISSDNAKNTVYLQMNSLKPEDTAVYYCAAGPSDPTLCGSLPDFGSWGQG TRVTVSS; and
CDR1 sequence is as set forth in SEQ ID NO: 23: GFTEDDYV;
CDR2 sequence is as set forth in SEQ ID NO: 24: MSSSDGIT;
CDR3 sequence is as set forth in SEQ ID NO: 25: AAGPSDPTLCGSLPDFGS.

### Example 6

### Synthesis and Characterization of [⁶⁸Ga]Ga-NOTA-ICOS-53

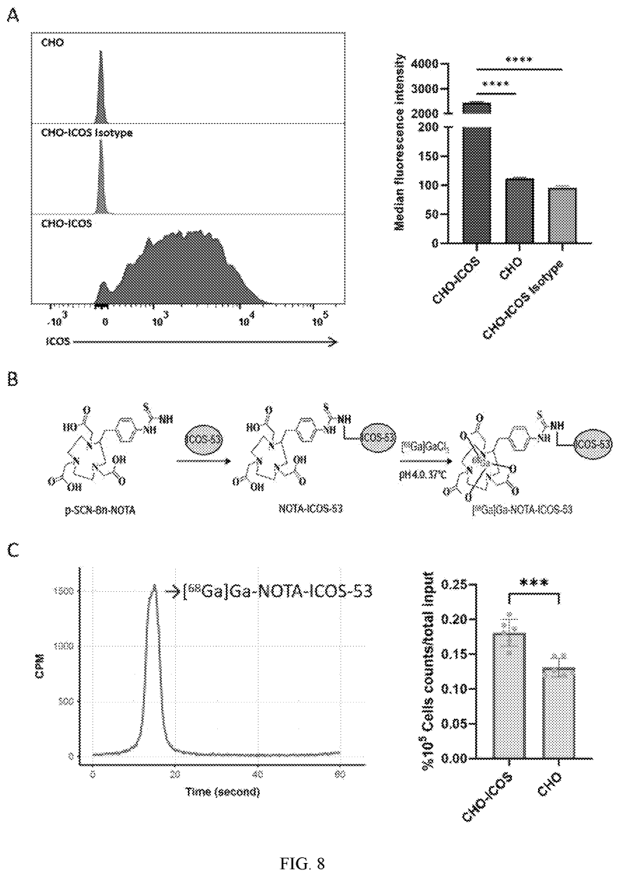

To verify the binding specificity of ICOS-53 to human ICOS protein, a CHO-ICOS cell line stably expressing human ICOS was used to conduct an in vitro cellular uptake study. Flow cytometry assay was performed on CHO-ICOS and CHO cells to confirm ICOS expression. As shown in FIG. 8A, the median fluorescence intensity (MFI) of ICOS on CHO-ICOS cells (2431.40±31.05) was much higher than that of the CHO group (111.20±1.48) and the isotype control group (95.62±3.13), P<0.0001.

As shown in FIG. 8B, the synthesis process of [⁶⁸Ga]Ga-NOTA-ICOS-53 was as follows: ICOS-53 was added to p-SCN-Bn-NOTA (NOTA is 1,4,7-triazacyclononane-1,4,7-triacetic acid) to obtain NOTA-ICOS-53; then [⁶⁸Ga]GaCl3 was added to NOTA-ICOS-53, followed by incubation at about 37 °C for 30 minutes to obtain [⁶⁸Ga]Ga-NOTA-ICOS-53.

As shown in FIG. 8C, radiochemical purity analysis showed that the radiochemical purity of [⁶⁸Ga]Ga-NOTA-ICOS-53 was >99%; compared with CHO cells (0.13±0.01%), higher radioactivity retention (0.18±0.02%) was detected in CHO-ICOS cells (P<0.001), indicating that [⁶⁸Ga]Ga-NOTA-ICOS-53 could specifically bind human ICOS protein.

### Example 7

### PET/CT Imaging Study of [⁶⁸Ga]Ga-NOTA-ICOS-53 in CHO-ICOS and CHO Tumor Models

To evaluate the ability of [⁶⁸Ga]Ga-NOTA-ICOS-53 to detect human ICOS protein in vivo, two tumor models, CHO-ICOS and CHO, were constructed, and PET/CT imaging studies were performed therein.

All studies involving animal subjects were approved by the Institutional Animal Care and Use Committee of Harbin Medical University. 5×10⁶ CHO cells and CHO-ICOS cells suspended in 50 μL of PBS were subcutaneously injected into the left shoulder and right shoulder of female NSG mice, respectively. Tumor volume was recorded with calipers every other day, and tumor volume was calculated using the formula (π/6)×length×width×height.

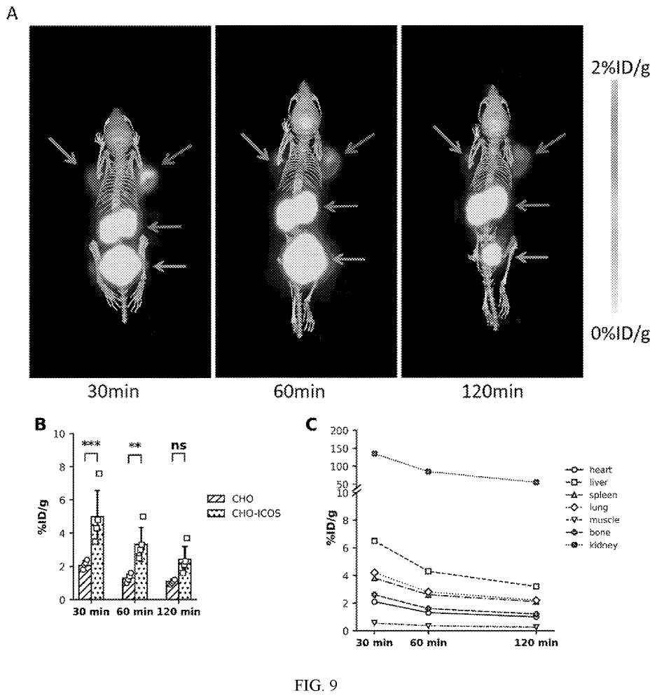

PET data were acquired at 30, 60, and 120 minutes after administration of [⁶⁸Ga]Ga-NOTA-ICOS-53. PET images are shown in FIG. 9A, and significant PET signals could be observed at the CHO-ICOS tumors (purple arrows) at all detection time points. To quantify tracer uptake in the two tumors, regions of interest (ROIs) were delineated in 3D mode. As shown in FIG. 9B, at all time points, tracer uptake (% ID/g) in CHO-ICOS tumors was significantly higher than that in CHO tumors (30 minutes: CHO-ICOS 4.99±1.59% ID/g, CHO 2.05±0.29% ID/g; 60 minutes: CHO-ICOS 3.32±1.02% ID/g, CHO 1.29±0.24% ID/g; 120 minutes: CHO-ICOS 2.43±0.77% ID/g, CHO 1.09±0.14% ID/g), indicating that [⁶⁸Ga]Ga-NOTA-ICOS-53 had specificity for ICOS-positive cells and tissues (n=4). To test the pharmacokinetics of this novel PET tracer, ROI quantification of major organs was also recorded, and the time-course pharmacokinetic curve is shown in FIG. 9C. Similar to most nanobody imaging studies, [⁶⁸Ga]Ga-NOTA-ICOS-53 was rapidly cleared from the blood pool and other major organs, which may be mediated by urinary excretion.

### Example 8

### Ex Vivo Biodistribution and Immunofluorescence Staining of [⁶⁸Ga]Ga-NOTA-ICOS-53

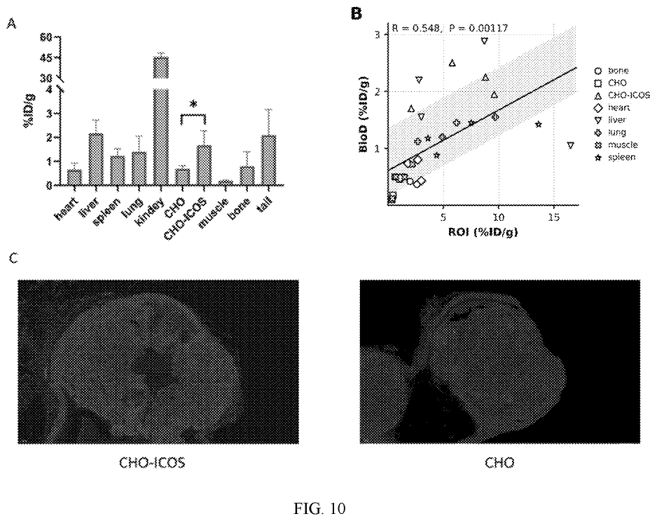

After the final PET scan was completed, all mice were immediately euthanized, and biodistribution studies were performed in major organs. As shown in FIG. 10A, compared with CHO tumors (0.70±0.12% ID/g), higher accumulation of [⁶⁸Ga]Ga-NOTA-ICOS-53 could be detected in CHO-ICOS tumors (1.67±0.60% ID/g) (P<0.05), which was consistent with the tumor ROI measurement results. The tumor-to-blood pool signal ratio in the CHO-ICOS group was higher than that in the CHO group (CHO-ICOS: 2.83±1.08, CHO: 1.17±0.35, P=0.0261).

To verify the accuracy of ROI measurements, further linear regression analysis was also performed. As shown in FIG. 10B, a good correlation between biodistribution quantification and PET ROI measurements was observed in all selected major organs (R=0.548, P=0.00117). As shown in FIG. 10C, the immunofluorescence staining experiment confirmed the difference in ICOS expression between CHO-ICOS and CHO tumors, indicating that the higher uptake of [⁶⁸Ga]Ga-NOTA-ICOS-53 in CHO-ICOS tumors should be attributed to the expression of ICOS protein.

In the examples herein, portions of the technical scope claimed by the present disclosure where midpoint values are not exhaustively described, and new technical solutions formed by equivalent replacement of a single technical feature or a plurality of technical features in the technical solutions of the examples, are likewise within the scope claimed by the present disclosure; meanwhile, in all listed or unlisted examples of the solutions of the present disclosure, the respective parameters in the same example merely represent an instance of the technical solution thereof (i.e., a feasible solution), and there is no strict coordination or limitation relationship among the respective parameters, wherein the respective parameters may be replaced with one another without violating axioms and the objectives of the present disclosure, unless otherwise specifically stated.

The technical means disclosed in the solutions of the present disclosure are not limited to the technical means disclosed above, and also include technical solutions consisting of any combination of the above technical features. The above describes specific embodiments of the present disclosure, and it should be noted that those of ordinary skill in the art may further make several improvements and modifications without departing from the principles of the present disclosure, and such improvements and modifications are also regarded as falling within the scope of protection of the present disclosure.

The specific examples described herein are merely illustrative of the spirit of the present disclosure. Those skilled in the art to which the present disclosure pertains may make various modifications or supplements to the described specific examples or replace them in a similar manner, without departing from the spirit of the present disclosure or exceeding the scope defined by the appended claims.

What is claimed is:

1. A polypeptide that binds human ICOS (Inducible T Cell Costimulator), comprising at least one VHH (Variable Heavy domain of Heavy chain), wherein the VHH comprising CDR1, CDR2, and CDR3, and a combination of the CDR1, CDR2, and CDR3 is selected from the group consisting of:
(a) a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
(d) a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22; and
(e) a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25.

2. The polypeptide according to claim 1, comprising an amino acid sequence selected from the group consisting of SEQ ID NO: 6, SEQ ID NO: 7, SEQ ID NO: 8, SEQ ID NO: 9, and SEQ ID NO: 10.

3. The polypeptide according to claim 1, wherein the CDR1 comprises the amino acid sequence set forth in SEQ ID NO: 11, the CDR2 comprising the amino acid sequence set forth in SEQ ID NO: 12, and the CDR3 comprising the amino acid sequence set forth in SEQ ID NO: 13.

4. The polypeptide according to claim 2, comprising the amino acid sequence set forth in SEQ ID NO: 6.

5. A polynucleotide encoding the polypeptide according to claim 1.

6. The polynucleotide according to claim 5, comprising a sequence selected from the group consisting of SEQ ID NO: 1, SEQ ID NO: 2, SEQ ID NO: 3, SEQ ID NO: 4, and SEQ ID NO: 5.

7. An expression vector comprising the polynucleotide according to claim 5.

8. A conjugate comprising the polypeptide according to claim 1, wherein the polypeptide is conjugated to a radionuclide.

9. The conjugate according to claim 8, wherein the radionuclide is ⁶⁸Ga.

10. A pharmaceutical composition comprising the conjugate according to claim 8 and a pharmaceutically acceptable carrier.

11. A method for detecting or imaging activated T cells, comprising:
administering the conjugate according to claim 8 to a sample; and
detecting a signal from the radionuclide.

12. A method for treating a tumor, comprising administering to a subject in need thereof a therapeutically effective amount of the polypeptide according to claim 1.


SEQUENCE LISTING (25 sequences)

SEQ ID NO: 1 - DNA, 388 bp, synthetic construct
  source 1..388: mol_type = other DNA
caggtgcagctcgtggagtctgggggaggcttggtgcagcctgggggttctctgagactctcctgtgcagcctctggattcacttttgagcgttggaccatggggtggttccgccagaccccagggaaggagcgcgaggggatctcatgtattagtagtagtgatggtagcacatactatggagactccaaatactatggagactccgttcggggccgattcactatttccagagacaacgccaagaacacagtatatctgcaaatgaacaacttgaaacctgaggacacggccgtttattactgtgcagcagcccaagggtatagcgactatggatgctacggcatggagtactggggcaaagggaccctggtcaccgtctcctcat

SEQ ID NO: 2 - DNA, 370 bp, synthetic construct
  source 1..370: mol_type = other DNA
caggtgcagctcgtggagtccggcggaggcttggtgcagcctggggggtctctgagactctcctgtgcagcctctggaaacatcttcagtatcaatgccatgggctggtaccgccaggctccagggaagcagcgcgagttggtcgcagcgtttactagtgatggtagcacagactatggagactccgtgaagggccgattcaccatctctagggacaacgccgagaacacggtggttttgcaaatgaacagcctgaaacctgaggacacggccgtctattactgcaatgcccaagttttctcaaggactatggagggctatgagggcatggagtactggggcaaagggaccctggtcaccgtctcctcag

SEQ ID NO: 3 - DNA, 370 bp, synthetic construct; identical to SEQ ID NO: 2
  source 1..370: mol_type = other DNA

SEQ ID NO: 4 - DNA, 394 bp, synthetic construct
  source 1..394: mol_type = other DNA
gaggtgcagctggtggagtctgggggaggcttggtgcagcctgggggttctctgagactctcctgtgcagcctctggagccactttggataattatgccacaggctggttccgccaggccccagggaaggagcgcgaggaggtctcatgtataagtggtagtggtgatggtaacacatactatgcagactccgtgaagggccgattcaccgtctccagagacagcgccaagaacacggtgtatctggaaatgaacagcctgaaaccggaggacacggccgtttattactgtgcagccgagggaccatatacatactatagtggtactggctgcccgggcacaacagggtatctcgaagtttggggccagggcaccctggtcactgtctcctcag

SEQ ID NO: 5 - DNA, 376 bp, synthetic construct
  source 1..376: mol_type = other DNA
gaggtgcagctggtggagtctgggggagacttggcgcaggctggggggtctttgagactctcctgtgcagcctctggattcactttcgatgattatgtcataggctggttccgccaggccccagggaaggagcgtgagggggtctcatgtatgagtagtagtgatggtataacatactacgcagactccgtgaagggccgattcaccatctccagtgacaacgccaagaatacggtgtatctgcaaatgaacagcctgaaacctgaggacacggccgtttattactgtgcagcgggacccagtgacccaacgctgtgcggtagcttacctgactttggttcctggggccaggggacccgggtcaccgtctcttcag

SEQ ID NO: 6 - protein, 129 aa, synthetic construct
QVQLVESGGGLVQPGGSLRLSCAASGFTFERWTMGWFRQTPGKEREGISCISSSDGSTYYGDSKYYGDSVRGRFTISRDNAKNTVYLQMNNLKPEDTAVYYCAAAQGYSDYGCYGMEYWGKGTLVTVSS

SEQ ID NO: 7 - protein, 123 aa, synthetic construct
QVQLVESGGGLVQPGGSLRLSCAASGNIFSINAMGWYRQAPGKQRELVAAFTSDGSTDYGDSVKGRFTISRDNAENTVVLQMNSLKPEDTAVYYCNAQVFSRTMEGYEGMEYWGKGTLVTVSS

SEQ ID NO: 8 - protein, 125 aa, synthetic construct
QLQLVESGGGLVQAGGSLRVSCAASGFTFDDYTIGWFRQSPGKEREGVSCISSSVGITYYADSVKGRFTISSDNAKNTVYLQMNSLKPEDTAVYYCAAGPTDSTLCGSLPDSGSWGQGTQVTVSS

SEQ ID NO: 9 - protein, 131 aa, synthetic construct
EVQLVESGGGLVQPGGSLRLSCAASGATLDNYATGWFRQAPGKEREEVSCISGSGDGNTYYADSVKGRFTVSRDSAKNTVYLEMNSLKPEDTAVYYCAAEGPYTYYSGTGCPGTTGYLEVWGQGTLVTVSS

SEQ ID NO: 10 - protein, 125 aa, synthetic construct
EVQLVESGGDLAQAGGSLRLSCAASGFTFDDYVIGWFRQAPGKEREGVSCMSSSDGITYYADSVKGRFTISSDNAKNTVYLQMNSLKPEDTAVYYCAAGPSDPTLCGSLPDFGSWGQGTRVTVSS

SEQ ID NO: 11 - protein, 8 aa, synthetic construct
GFTFERWT

SEQ ID NO: 12 - protein, 14 aa, synthetic construct
ISSSDGSTYYGDSK

SEQ ID NO: 13 - protein, 16 aa, synthetic construct
AAAQGYSDYGCYGMEY

SEQ ID NO: 14 - protein, 8 aa, synthetic construct
GNIFSINA

SEQ ID NO: 15 - protein, 7 aa, synthetic construct
FTSDGST

SEQ ID NO: 16 - protein, 17 aa, synthetic construct
NAQVFSRTMEGYEGMEY

SEQ ID NO: 17 - protein, 8 aa, synthetic construct
GFTFDDYT

SEQ ID NO: 18 - protein, 8 aa, synthetic construct
ISSSVGIT

SEQ ID NO: 19 - protein, 18 aa, synthetic construct
AAGPTDSTLCGSLPDSGS

SEQ ID NO: 20 - protein, 8 aa, synthetic construct
GATLDNYA

SEQ ID NO: 21 - protein, 9 aa, synthetic construct
ISGSGDGNT

SEQ ID NO: 22 - protein, 23 aa, synthetic construct
AAEGPYTYYSGTGCPGTTGYLEV

SEQ ID NO: 23 - protein, 8 aa, synthetic construct
GFTFDDYV

SEQ ID NO: 24 - protein, 8 aa, synthetic construct
MSSSDGIT

SEQ ID NO: 25 - protein, 18 aa, synthetic construct
AAGPSDPTLCGSLPDFGS